(19) United States
(12) Patent Application Publication
(10) Pub. No.: US 2017/0100059 A1
(43) Pub. Date: Apr. 13, 2017

(54) LUNG FUNCTION MONITORING

(71) Applicant: GENERAL ELECTRIC COMPANY, Schenectady, NY (US)

(72) Inventors: Tzu-Jen KAO, Watervliet, NY (US); David Michael DAVENPORT, Niskayuna, NY (US); Jeffrey Michael ASHE, Gloversville, NY (US); Bruce Courtney Campbell AMM, Clifton Park, NY (US)

(73) Assignee: GENERAL ELECTRIC COMPANY, Schenectady, NY (US)

(21) Appl. No.: 14/879,133

(22) Filed: Oct. 9, 2015

(51) Int. Cl. A61B 5/08; A61B 5/00; A61B 5/091; A61B 5/053

(52) U.S. Cl. CPC A61B 5/0809; A61B 5/0536; A61B 5/6823; A61B 5/091; A61B 5/742; A61B 2560/0223

(57) ABSTRACT

A signal of a current-voltage sensor array in one embodiment is processed for output of data in a form for use by a care provider. The current-voltage sensor array in one embodiment is a non-invasive current-voltage sensor array adapted to surround a thorax of a patient. The output of data in one embodiment is provided in a lung function visualization output form that depicts functioning of a patient's lungs.

### STATEMENT REGARDING FEDERALLY SPONSORED RESEARCH & DEVELOPMENT

This invention was made with Government support under contract number 1R01HL109854 awarded by the National Institute of Health. The Government has certain rights in the invention.

### FIELD

The disclosure relates to physiological monitoring in general and in particular to lung function monitoring.

### BACKGROUND

Electrical Impedance Tomography (EIT) is an imaging modality that estimates the electrical properties at the interior of region of interest (ROI) from measurements made on its surface. It is also a noninvasive, radiation free technology and could be built as a low-cost and portable device. Typically, small electrical currents or voltages, or both, are injected into the object through electrodes placed on its surface and the corresponding voltages (or currents) are measured. Then, the reconstruction algorithms use the knowledge of the excitation patterns and the resulting measurements to solve the inverse problem of computing the conductivity and permittivity distributions in the object and displaying the result in 2-D or 3-D images. Most current EIT devices in clinical study and publications in the field use a single current generator with a multiplexer to drive a constant current (5 mA˜12 mA) on a pair of electrodes (adjacent electrodes or opposite electrode) at a particular kHz frequency and then measure the corresponding voltages on the other electrodes.

### BRIEF DESCRIPTION

A signal of a current-voltage sensor array in one embodiment is processed for output of data in a form for use by a care provider. The current-voltage sensor array in one embodiment is a non-invasive current-voltage sensor array applied to surround a thorax of a patient. The output of data in one embodiment is provided in a lung function visualization output form that depicts functioning of a patient's lungs.

### DRAWINGS

FIG. 1 is block diagram of an apparatus having a current-voltage sensor array and a processing circuit for processing signals of the current-voltage sensor array;

FIG. 2 is a diagram illustrating a current-voltage sensor array append to a patient;

FIG. 3 is an example of a lung function visualization output form based processing of a signal of a current voltage sensor array;

FIG. 4 is an example of a lung function visualization output form based on processing of a signal of a current-voltage sensory array;

FIG. 5 is an example of a lung function visualization output form based on processing of a signal of a current-voltage sensor array;

FIG. 6 is an example of a lung function visualization output form based on processing of a signal of a current-voltage sensor array;

FIG. 7 is an example of a lung function visualization output form based on processing of a signal of a spirometer;

FIG. 8 is an example of a lung function visualization output form based on processing of a signal of a spirometer;

FIG. 9 is an example of a lung function visualization output form provided by a flow volume loop in various stages corresponding to various patient conditions based on processing a signal of a current-voltage sensor array;

FIG. 10 is an example of a lung function visualization output form provided by a flow volume loop in various stages corresponding to various patient conditions based on processing of a signal of a spirometer;

FIG. 11 illustrates a lung function visualization output form subject to processing for providing a respiratory index lung function visualization output form based on processing a signal of a current-voltage sensor array;

FIG. 12 illustrates a lung function visualization output form subject to processing for providing a respiratory index lung function visualization output form based on processing a signal of a current-voltage sensor array;

FIG. 13 illustrates a respiratory index lung function visualization output form based on processing a signal of a current-voltage sensor array;

FIG. 14 illustrates a lung function visualization form subject to processing for providing a respiratory index lung function visualization output form based on processing of a signal of a spirometer;

FIG. 15 illustrates a lung function visualization form subject to processing for providing a respiratory index lung function visualization output form based on processing of a signal of a spirometer;

FIG. 16 illustrates a respiratory index lung function visualization output form based on processing of a signal of a spirometer;

FIG. 17A-17J illustrate examples of lung function visualization output forms based on a processing of a signal of a current-voltage sensor array;

FIG. 18 is a flow diagram illustrating a method using a current-voltage sensor array for output of data according to a lung function visualization output form; and

FIG. 19 is a flow diagram illustrating a method using a current-voltage sensor array for output of data according to a lung function visualization output form.

### DETAILED DESCRIPTION

A signal of a current-voltage sensor array in one embodiment is processed for output of data in a format for use by a care provider. The current-voltage sensor array in one embodiment is a non-invasive current-voltage sensor array adapted to surround a thorax of a patient. The output of data in one embodiment is provided in a lung function visualization output form.

An exemplary apparatus 10 for performance of lung function monitoring is shown in FIG. 1. Apparatus 10 can include a current-voltage sensor array 100 and a signal processing circuit 200.

Current-voltage sensor array 100 in one embodiment can include first electrode apparatus 110 and second electrode apparatus 110 and in one embodiment can include additional electrode apparatus 110. Each electrode apparatus 110 can include an electrode. In one embodiment, current-voltage sensor array 100 is operative so that current is driven through a first electrode apparatus 110 using a current source and voltage is measured at a second electrode apparatus 110 using a voltage meter. In one embodiment, current-voltage sensor array 100 is operative so that voltage is applied at a first electrode apparatus 110 using a voltage source and current is measured at a second electrode apparatus 110 using a current meter. In one embodiment, one or more electrode apparatus 110 can include an on board current source and/or an on board voltage source and/or an on board current meter and/or and on board voltage meter. In one embodiment, current-voltage sensor array 100 can include multiplexing circuitry so that one or more of a current source, voltage source, current meter, or voltage meter is shared between two or more electrode apparatus.

In one embodiment, signal processing circuit 200 is processor based. In one embodiment, signal processing circuit 200 can include one or more input-output interface device 210 for communication with, e.g., current-voltage sensor array 100, and/or one or more external processing circuits. Signal processing circuit 200 can also include one or more central processing unit (CPU) 212, one or more memory device 214 one or more storage device 216 and one or more output device 220. One or more output device 220 in one embodiment is, e.g., a display with or without an associated touch screen. Devices 210, 212, 214, 216, and 220 in one embodiment are in communication via a system bus 218. Signal processing circuit 200 can output data to a bus connected output device 220 and/or to an external output device configured in the manner of output device 220 which in one embodiment is in communication with signal processing circuit 200 via input-output interface device 210. The external output device in one embodiment is, e.g., a display device of, e.g., a personal computer, laptop computer, tablet computer, or mobile phone.

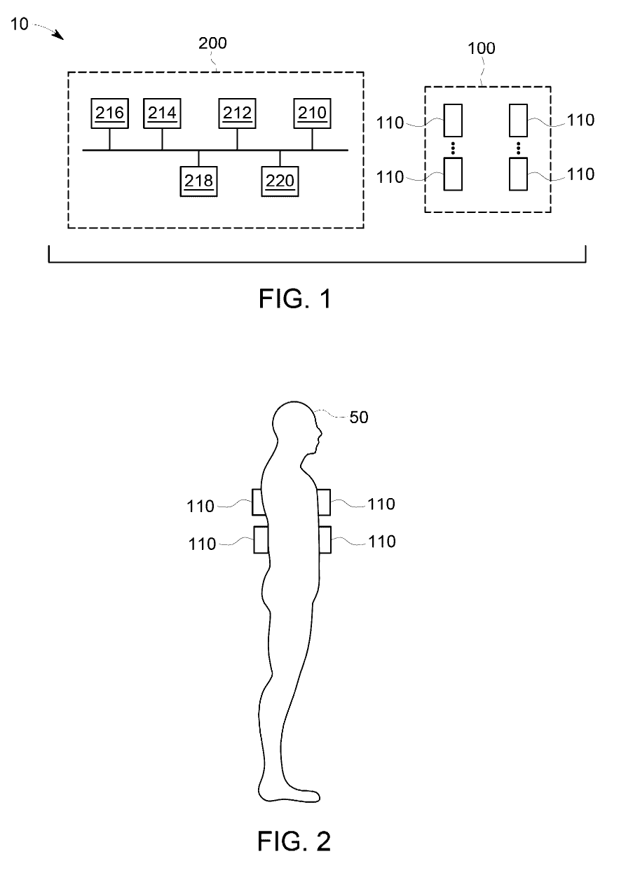

Signal processing circuit 200 can process signal of a current-voltage sensor array 100 for output of data on output device 220 according to a lung function visualization output form. A signal output by current-voltage sensor array 100 in one embodiment is in response to an excitation signal generated by current-voltage sensor array 100 based on a control signal sent from signal processing circuit 200 to current-voltage sensor array 100. Current-voltage sensor array 100 in one embodiment is adapted to surround a thorax of a patient 50 as is shown in FIG. 2. In one embodiment, apparatus 10 having current-voltage sensor array 100 is incorporated as part of an Electrical Impedance Tomography (EIT) imaging system.

In one embodiment, apparatus 10 having current-voltage sensor array 100 and signal processing circuit 200 is incorporated as part of a single source Electrical Impedance Tomography (EIT) imaging system. A current-voltage sensor array 100 of a single source EIT imaging system in one embodiment is controlled through appropriate multiplexing circuitry so that at a given time a single source (current or voltage) is controlled to drive a single electrode apparatus. Examples of a single source EIT imaging systems include the SHEFFIELD MK 3.5 available from Maltron International, the GOE MF II available from Carefusion, the ENLIGHT 1800 available from Timpel SA, the PULMOVISTA 500 available from Draeger Medical, the SWISSTOM BB2 available from Swisstom AG.

In one embodiment, apparatus 10 having current-voltage sensor array 100 and signal processing circuit 200 is included as part of a multiple source Electrical Impedance Tomography (EIT) imaging system. A current-voltage sensor array 100 of a multiple source EIT imaging system in one embodiment is controlled that at a given time multiple sources (current or voltage) are controlled to drive multiple electrode apparatus. Examples of multiple source EIT imaging systems described in the academic literature include the GENESIS system of General Electric, ACT3 and ACT4 systems of Rensselaer Polytechnic Institute, the BROOKES system of Oxford University, and IIRC system of Hyung Hee University. Aspects of EIT Imaging systems are described in A High Precision Parallel Current Drive Experimental System, Proceedings of the 15th International Conference on the Biomedical Applications of Electrical Impedance Tomography, Ontario, CA (2014), An Electric Current Tomograph, IEEE Transactions on Biomedical Engineering, Vol. 35, No. 10 (1988), Electrical Impedance Tomography Methods, History and Applications, Institute of Physics Publishing ltd (2005).

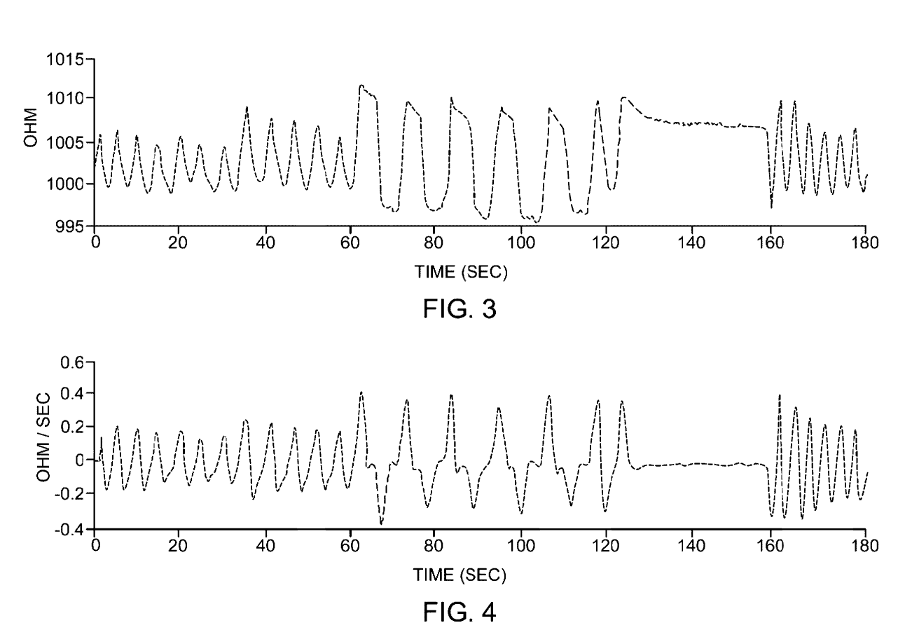

In one embodiment, signal processing circuit 200 can obtain a signal of current-voltage sensor array 100, process the signal and output, e.g., on output device 220 a lung function visualization output form. In FIG. 3 there is shown an example of a lung function visualization output form determined based on a processing of a signal obtained from current-voltage sensor array 100. As shown in FIG. 3 a lung function visualization output form in one embodiment is an impedance change waveform lung function visualization output form indicating impedance change over time. The impedance change waveform of FIG. 3 in one embodiment is provided based on an output signal of current-voltage sensor array 100. Generally, signal processing circuit 200 can generate an impedance change waveform lung function visualization output form, such as that illustrated by FIG. 3, by dividing voltage by current at each point in time for each electrode apparatus 110 and each excitation signal. For output of a global impedance measurement, as illustrated in the particular embodiment of FIG. 3, signal processing circuit 200 can select and sum the total to compute impedance for each point in time. In one aspect, a subset of excitation patterns and/or a subset of electrode apparatuses 110 to be included in the summation, in order to make the impedance change waveform lung function visualization output form more sensitive or less sensitive to particular regions in a patient (e.g., more sensitive to a patient's lungs, less sensitive to chest or abdomen, etc.) As will be set forth in greater detail herein it was determined that changes in impedance in one embodiment are proportional to volume changes of air within a lung. As more air enters the lungs, impedance of the lungs increases. Accordingly, it was determined that changes in a parameter provided using a current-voltage sensor array 100 such as impedance can be used to represent changes in volume of a lung. Referring to FIG. 3, positive peaks of the waveform output form of FIG. 3 refer to inspiration periods (lungs filled, higher impedance), and negative peaks of the waveform output form of FIG. 3 refer to expiration periods (lungs emptied, lower impedance). A waveform having impedance information herein can be regarded to include admittance information. Admittance is reciprocated to impedance.

Another lung function visualization output form is shown in FIG. 4. As shown in the embodiment of FIG. 4, a lung function visualization output form based on a processing of a signal obtained from current-voltage sensor array 100 in one embodiment is provided by an impedance change rate waveform visualization output form indicating impedance change rate over time. Signal processing circuit 200 can provide an impedance change rate waveform visualization output form as depicted in FIG. 4 by determining of a first derivative of an impedance change waveform, a visualization output form of which is shown in FIG. 3. It was determined that an impedance change rate can be proportional to volume change rates. Accordingly, it was determined that an impedance change rate can be used to represent lung volume change rate.

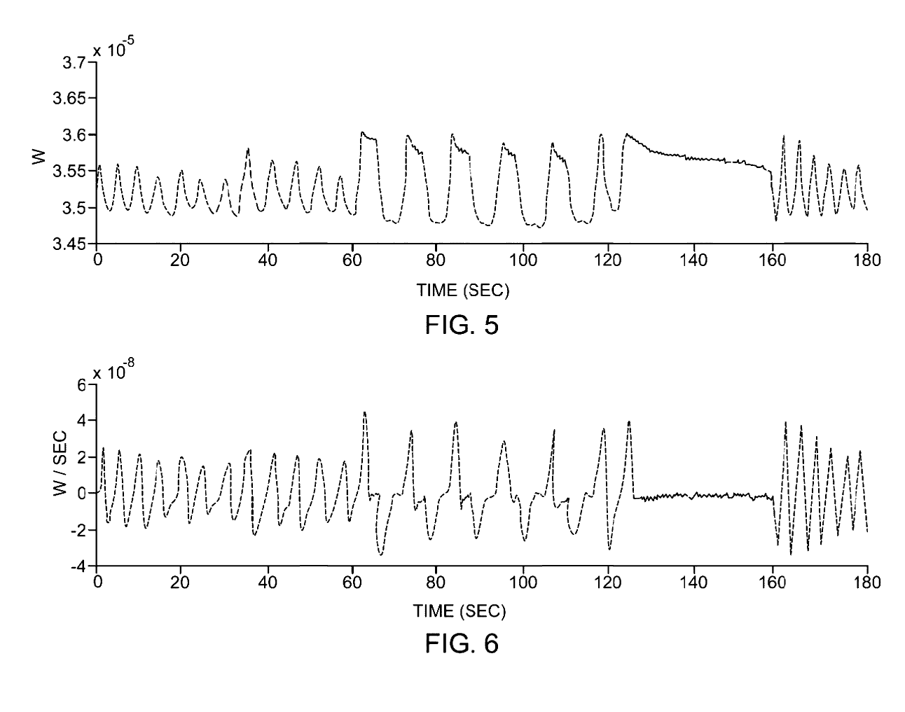

In one embodiment, as shown in FIG. 5, a lung function visualization output form determined by signal processing circuit 200 based on a processing of a signal of a current-voltage sensor array 100 is a power change waveform lung function visualization indicating power change over time. In FIG. 5, the X-axis represents time (e.g., in seconds) and the Y-axis may represent power. Generally, signal processing circuit 200 can generate a power change waveform lung function visualization output form, such as that illustrated by FIG. 5, by multiplying measured voltage (or applied voltage) by applied current (or measured current) at each point in time for each electrode apparatus 110 and each excitation. For output of a global power measurement as depicted in FIG. 5, signal processing circuit 200 can select and sum the total to compute power for each point in time. In one aspect, a subset of excitation patterns and/or a subset of electrode apparatuses 110 can be included in the summation, in order to make the power change waveform lung function visualization output form more sensitive or less sensitive to particular regions in a patient (e.g., more sensitive to a patient's lungs, less sensitive to chest or abdomen, etc.) As will be set forth in greater detail herein it was determined that changes in power can be proportional to volume changes of air. Accordingly, it was determined that changes in a parameter provided using a current-voltage sensor array 100, such as power can be used to represent changes in volume of a lung.

Another lung function visualization output form is shown in FIG. 6. As shown in the embodiment of FIG. 6, a lung function visualization output form based on a processing of a signal obtained from current-voltage sensor array 100 in one embodiment is provided by a power change rate waveform visualization output form indicating power change rate over time. Signal processing circuit 200 can provide a power change rate waveform visualization output form as depicted in FIG. 6 by determining of a first derivative of a power change waveform, a visualization output form of which is shown in FIG. 5. It was determined that power change rate can be proportional to volume change rate. Accordingly, it was determined that a power change rate can be used to represent lung volume change rate.

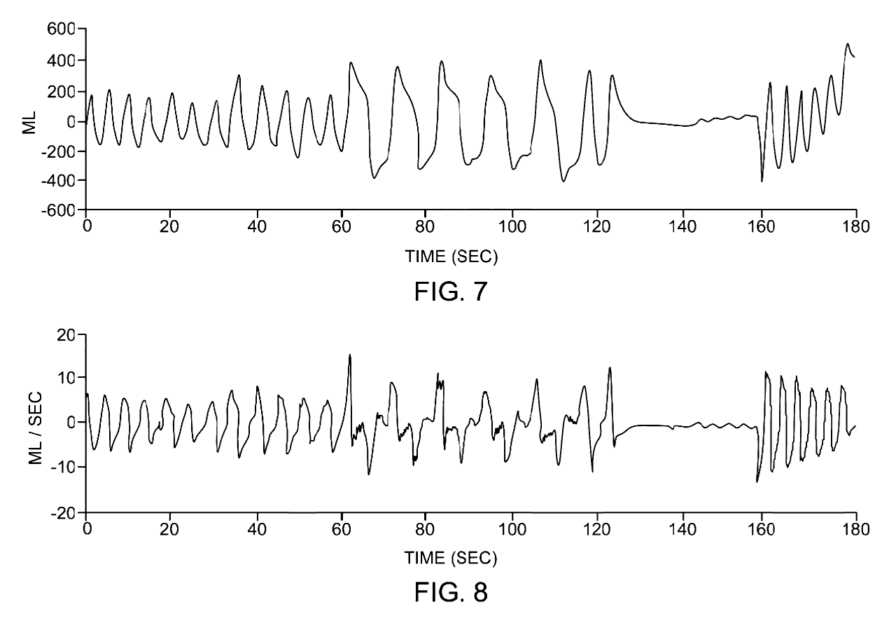

Referring to FIG. 7-8 a lung function visualization output form can be provided by processing circuit 200 based on a processing of a signal obtained from a spirometer. A spirometer can sense movement of air into out of patient lungs using, e.g., a pressure transducer or an ultrasonic transducer. Referring to FIG. 7 there is illustrated a lung function visualization output form provided by processing circuit 200 based on a processing of a signal obtained from a spirometer provided by a pneumotachograph spirometer. The output forms of FIGS. 3-4 and FIGS. 5-6 have the same general waveform shapes as the output forms as shown in FIGS. 7 and 8 (noting that P=I²R small differences in shape between an impedance change waveform and a power change waveform can be attributable to an impedance of a thorax deviating from a purely resistance impedance). Accordingly the current-voltage sensor array derived output forms of FIGS. 3 and 4 and FIGS. 5 and 6 can be regarded to simulate the spirometer derived output forms of FIGS. 7 and 8. Referring to FIG. 7, a lung function visualization output form as shown in FIG. 7 can be provided by integrating the output shown in FIG. 8. Where a spirometer is a pneumotachograph spirometer, an output signal can have an output form having the general shape of FIG. 8. Where a spirometer is a non-pneumotachograph spirometer, an output signal can have an output form having the general shape of FIG. 7 and an output form as shown in FIG. 8 can be provided by taking the first derivative of an output form having the general shape as shown in FIG. 7. It was determined that impedance of a patient's lungs and power dissipation through a patient's lungs can be proportional to air volume of a patient's lungs. Because impedance and power can be proportional to an air volume of patient's lungs it was determined that lung function visualization output forms provided based on a processing of a signal obtained from a current-voltage sensor array 100 can simulate lung function visualization output forms based on a processing of a signal obtained from a spirometer that measures lung air movement. Providing visualization output forms based on a processing of a signal obtained from a current-voltage sensor array 100 can be advantageous for various reasons. Current-voltage sensor array 100 in one embodiment is non-invasive and may not require any claustrophobia causing element wearable at a facial area of a patient. A breathing tube in some instances can alter a breathing pattern of a patient. In addition, use of a current-voltage sensor array 100 can facilitate output forms that illustrate lung functioning within a localized regions of a thorax.

In one embodiment, lung function visualization output forms as shown in FIGS. 3-6 which can be provided by signal processing circuit 200 based on a processing of a signal of current-voltage sensor array 100 can simulate a lung function visualization output form that in one embodiment is provided based on a processing of a signal obtained from a spirometer. The lung function visualization output forms of FIGS. 3 and 4 provided based on a processing of a signal obtained from current-voltage sensor array 100 can simulate a visualization output form as shown in FIG. 7-8 provided based on a processing of a signal obtained from a spirometer. The lung function visualization output forms of FIGS. 5 and 6 provided based on a processing of a signal obtained from current-voltage sensor array 100 can simulate a visualization output form as shown in FIG. 7-8 provided based on a processing of a signal obtained from a spirometer.

The impedance and power change waveform of FIGS. 3 and 5 can simulate a volume change waveform lung function visualization output form as shown in FIG. 7 provided based on a processing of a signal obtained from a spirometer, in which the X axis represents time (generally in seconds) and the Y axis represents volume (generally in liters or milliliters). The impedance change rate waveform of FIG. 4 and the power change rate waveform of FIG. 6 can simulate a volume change rate waveform lung function visualization output form as shown in FIG. 8 provided based on a processing of a signal obtained from a spirometer, in which the X axis represents time (generally in seconds) and the Y axis represents volume/time (generally in liters/sec. or milliliters/sec.). The volume change waveform lung function visualization output form in one embodiment is provided using a breathing tube with a spirometer, as may be commonly used in administering care to patients. A breathing tube with a spirometer in one embodiment is incorporated as part of a ventilator machine. A spirometer may be defined as an air volume measuring tool.

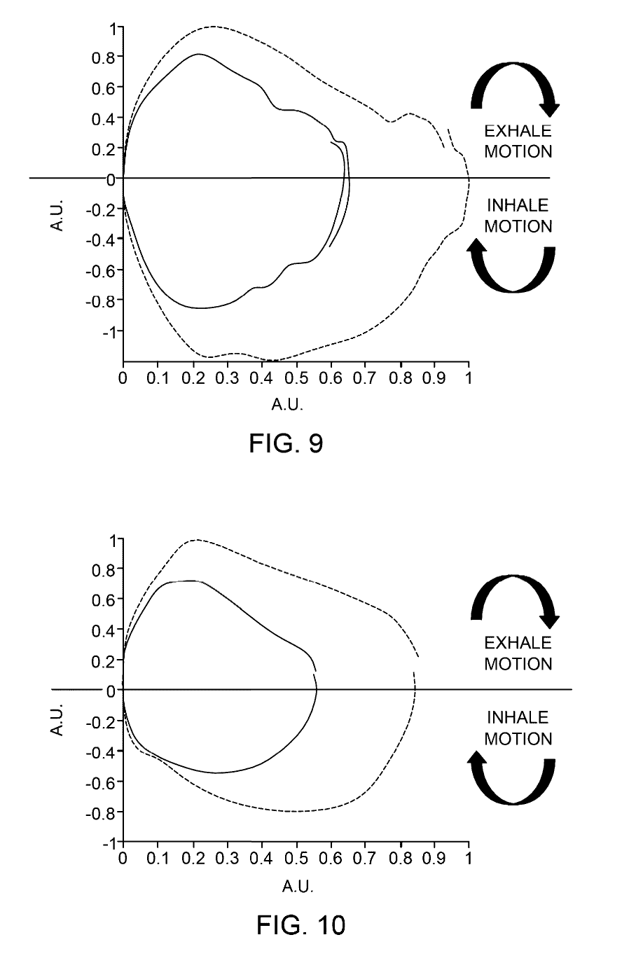

In one embodiment, as shown in FIG. 9, a lung function visualization output form that can be determined based on a processing of a signal of a current-voltage sensor array 100 can be a flow volume loop lung function visualization output form.

The flow volume loop lung function visualization output form as shown in FIG. 9 in one embodiment is determined and generated by signal processing circuit 200 as described below. A flow volume loop in one embodiment is determined based on impedance or power which may be calculated based on measurements of current and voltage using current-voltage sensor array 100, as discussed previously. Though flow need not be measured using a spirometer, the output of FIG. 9 can be regarded as a flow volume loop lung function visualization output form since as set forth herein, impedance or power can serve as an indicator of air volume. Using impedance as an example, impedance data can be obtained and segmented for each breath, in which a breath is defined as starting at end-expiration which is a local minimum of impedance, continuing through end-inspiration which is a local maximum of impedance, and ending at end-expiration (local minimum of impedance) which is the start of the next breath. The impedance data and the time derivative of impedance can be plotted on X and Y axes, where the X-axis represents volume (which is proportional to impedance) and the Y-axis represents air flow, which is proportional to the time derivative of impedance. Impedance data can thus be plotted relative to the X-axis and the time derivative of impedance is plotted relative to the Y-axis, starting at the graph's origin (0, 0) and proceeding clockwise for each breath cycle and returning to the origin at the end of each breath cycle. Using power as an example, power data can be obtained and segmented for each breath, in which a breath is defined as starting at end-expiration which is a local minimum of power, continuing through end-inspiration which is a local maximum of power, and ending at end-expiration (local minimum of power) which is the start of the next breath. The power data and the time derivative of power are plotted on X and Y axes, where the X-axis represents volume (which is proportional to power) and the Y-axis represents air flow, which is proportional to the time derivative of power. Power data can thus be plotted relative to the X-axis and the time derivative of power is plotted relative to the Y-axis, starting at the graph's origin (0, 0) and proceeding clockwise for each breath cycle and returning to the origin at the end of each breath cycle. In FIG. 9, the X axis is in units of power (normalized) and the Y axis is in units of time derivative of power (normalized). FIG. 9 can be provided using the data of FIGS. 5 and 6 in one example. However, as noted, the flow volume loop can alternatively be provided using current voltage sensor array by processing impedance data rather than power data. An output form having the shape of FIG. 9 can be provided using the data of FIGS. 3 and 4 in one example.

The lung function visualization output form of FIG. 9, which is a flow volume loop generated by signal processing circuit 200 based on measurements of current and voltage using current-voltage sensor array 100, can simulate a flow volume loop lung function visualization output as shown in FIG. 10. In the view of FIG. 10, the flow volume loop output can be determined using a breathing tube apparatus with a spirometer. FIG. 10 can be provided using the data of FIGS. 7 and 8 in one example. The X-axis can be provided in units of volume (normalized), and the Y axis can be provided in units of volume/time (normalized).

The flow volume loop lung function visualization output form as shown in FIG. 9 or 10 provides a variety of useful information. A flow volume loop diagram curve can be particularly helpful, for example, because a flow volume loop may contain speed information. As another example, the area of the curve, and particular the area of each curve as measured for each breath cycle, provides information on changes in flow and/or volume. An increase in the horizontal diameter of the curve, for instance, generally indicates an increase in breath volume. An increase in the vertical diameter, for instance, generally indicates an increase in flow. As well, the shape of the curve may provide useful information for health care providers about a patient. The top half of the flow volume loop represents the exhalation portion of the breath cycle, while the bottom half represents the inhalation portion. When a patient is intubated (breathing assisted by mechanical ventilator), the inhalation is forced or supported by the ventilator while exhalation occurs naturally. When the patient is no longer intubated, both inhalation and exhalation occur naturally. Comparing the shape of the inhalation curve during intubation with the shape of the curve following extubation can provide valuable information about a patient's progress, as the intubation curves may provide a reference point for subsequent observation.

An advantage of the flow-volume loop is that it can show whether airflow is appropriate for a particular lung volume. For example, airflow is normally slower at low lung volumes because elastic recoil is lower at lower lung volumes. Patients with pulmonary fibrosis have low lung volumes and their airflow appears to be decreased if measured alone. However, when airflow is presented as a function of lung volume, it becomes apparent that airflow is actually higher than normal (as a result of the increased elastic recoil characteristic of fibrotic lungs). Description of representative flow volume loop stages and corresponding patient conditions is as follows: (A) Normal. Inspiratory limb of loop is symmetric and convex. Expiratory limb is linear. Airflow at the midpoint of inspiratory capacity and airflow at the midpoint of expiratory capacity are often measured and compared. Maximal inspiratory airflow at 50% of forced vital capacity (MIF 50% FVC) is greater than maximal expiratory airflow at 50% FVC (MEF 50% FVC) because dynamic compression of the airways occurs during exhalation; (B) Obstructive disorder (e.g., emphysema, asthma). Although all airflow is diminished, expiratory prolongation predominates, and MEF<MIF. Peak expiratory flow is sometimes used to estimate degree of airway obstruction but depends on patient effort. (C) Restrictive disorder (e.g., interstitial lung disease, kyphoscoliosis). The loop is narrowed because of diminished lung volumes. Airflow is greater than normal at comparable lung volumes because the increased elastic recoil of lungs holds the airways open; (D) Fixed obstruction of the upper airway (e.g., tracheal stenosis, goiter). The top and bottom of the loops are flattened so that the configuration approaches that of a rectangle. Fixed obstruction limits flow equally during inspiration and expiration, and MEF=MIF; (E) Variable extrathoracic obstruction (e.g., unilateral vocal cord paralysis, vocal cord dysfunction). When a single vocal cord is paralyzed, it moves passively with pressure gradients across the glottis. During forced inspiration, it is drawn inward, resulting in a plateau of decreased inspiratory flow. During forced expiration, it is passively blown aside, and expiratory flow is unimpaired. Therefore, MIF 50% FVC<MEF 50% FVC; (F) Variable intrathoracic obstruction (e.g., tracheomalacia). During a forced inspiration, negative pleural pressure holds the floppy trachea open. With forced expiration, loss of structural support results in tracheal narrowing and a plateau of diminished flow. Airflow is maintained briefly before airway compression occurs. As set forth herein, a flow volume loop lung function visualization output form in one embodiment is provided by signal processing circuit 200 based on a processing of a signal obtained from a current-voltage sensor array 100 and/or a spirometer.

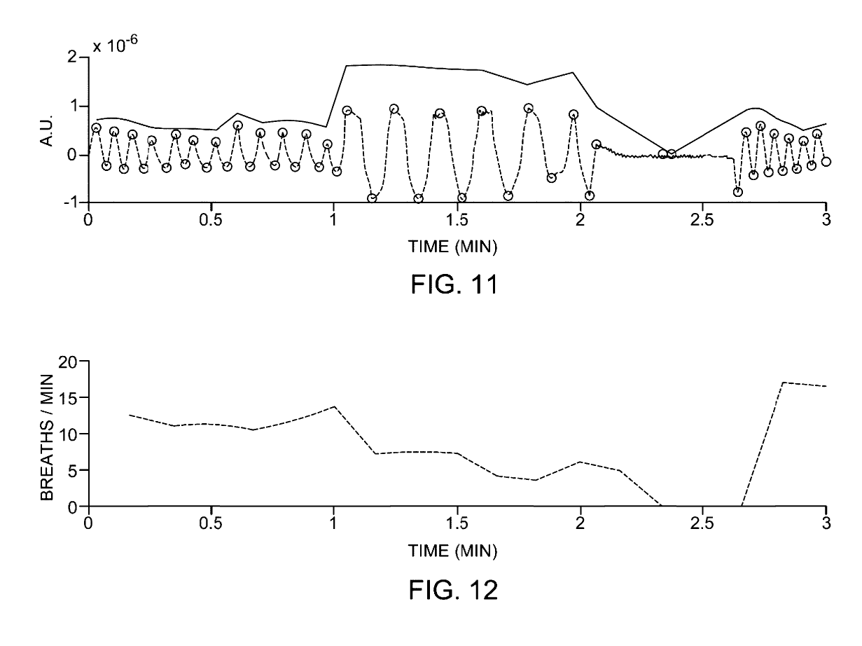

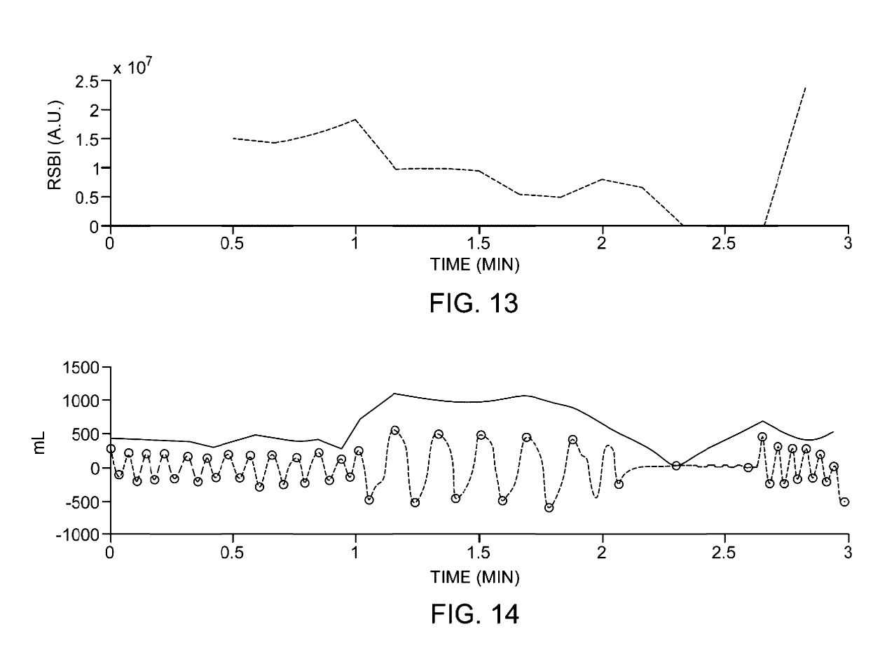

In one embodiment, as shown in FIG. 11-13, a lung function visualization output form that can be determined by signal processing circuit 200 based on a processing of a signal of a current-voltage sensor array 100 can be a respiratory index lung function visualization output form. In exemplary embodiments, a respiratory index lung function visualization output form can be a breaths per minute output form as shown in FIG. 12 or can be a rapid shallow breathing index (RSBI) lung function visualization output form, as illustrated in FIG. 13. A respiratory index, and in particular an RSBI, may be defined as a patient's breathing rate (usually expressed in breaths per minute) divided by the tidal volume (the amount of air exhaled during one breath cycle, usually expressed in liters per breath). Respiratory indices are thus measured in number of breaths per liter per minute. As discussed further below, any of the metrics that may be obtained using current-voltage sensor array 100 (such as impedance, power or admittance) may be used to calculate air volumes, and such calculations may, in exemplary embodiments, include calibrating the metrics obtained from current-voltage sensor array 100 according to a specific patient. Processing which can employed by signal processing circuit 200 for output of a respiratory index lung function visualization output form is described with reference to FIG. 11. Initially, a peak detection algorithm can be run for detections of peaks (indicated by circles). Further processing can be performed for determination of an envelope, and the waveform can be subject to a 10 second averaging time window for output of the breaths/min. waveform output form as shown in FIG. 12. For determination of the RSBI waveform output form as shown in FIG. 13 units of FIG. 11 and FIG. 12 can be normalized.

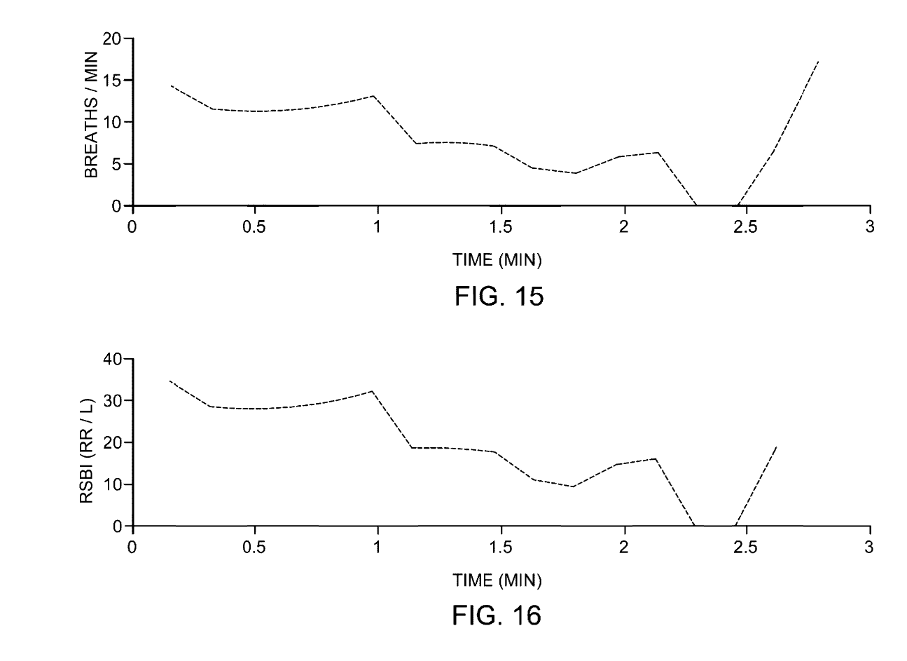

A breathing index lung function visualization data output form provided by processing in output of current-voltage sensor array 100 can simulate a breathing index lung function visualization output form provided by processing an output of a spirometer. In FIG. 14 there is illustrated that the spirometer derived output form of FIG. 7 can be subject to processing for determining positive and negative peaks (circles) and determination of an envelope. Referring to FIG. 15, a 10 second time averaging window can be applied for determination of a breaths per minute breathing index lung function visualization output form. Referring to FIG. 16 the breaths/min. units of FIG. 15 can be normalized for determination of the RSBI output form of FIG. 16.

The lung function visualization output form of FIGS. 12-13, which are respiratory index output forms provided by a breaths per minute (FIG. 12) and an RSBI (FIG. 13) lung function visualization output form, can simulate respiratory index lung function visualization output format provided based on a processing of a signal obtained from a spirometer as shown in FIGS. 15 and 16. A respiratory index lung function visualization output form may be used by health care providers and has a variety of useful information. In general, healthier patients have a lower respiratory rate and larger tidal volume, while patients in poorer condition may have a higher respiratory rate and smaller tidal volume. Thus, a lower respiratory index, e.g., RSBI value generally may indicate a healthier patient. Although opinions differ on what the threshold difference between healthier and sicker patients should be, generally an RSBI value greater than 65 breaths per liter per minute indicates a patient in poor condition. Providing a respiratory index output form with the breaths/min. index (FIG. 12) and/or RSBI output form as shown in FIG. 13, using a current-voltage sensor array 100, can provide numerous advantages, as set forth herein.

The lung function visualization output forms as shown in FIGS. 3-6, 9 and 11-13 which can be provided by a signal processing circuit 200 based on a processing of a signal of current-voltage sensor array 100 can simulate lung function visualization output forms that can be output using a breathing tube apparatus having a spirometer, such as the lung function visualization output forms illustrated in FIGS. 7-8, 10, 14-16, respectively. A breathing tube apparatus having a spirometer can be provided, for example, as part of a mechanical ventilator machine or, in another example, a standalone portable apparatus. Providing the lung function visualization output forms of one or more of FIGS. 7-8, 10, 14-16 can be advantageous in one aspect because a current-voltage sensor array 100 can be noninvasive and can cause a minimal amount of discomfort to a patient equipped with current-voltage sensor array 100.

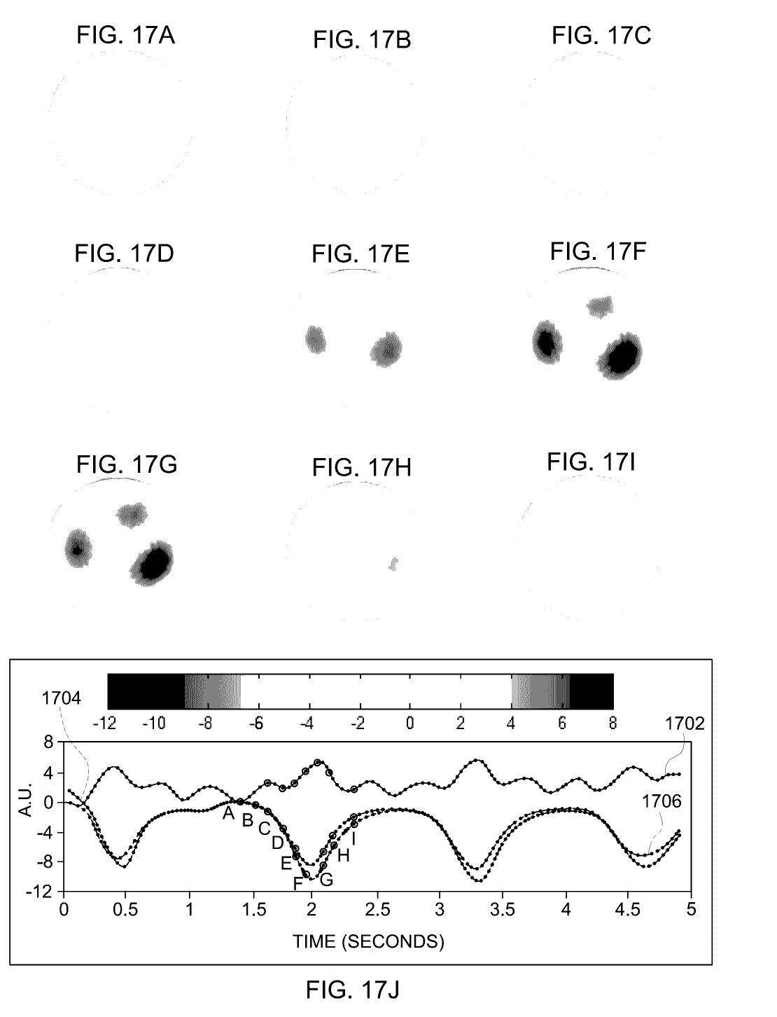

In another aspect, providing a lung function visualization output form by processing of a signal of a current-voltage sensor array 100 can facilitate visualization of lung functioning within specific localized regions of a patient thorax. FIGS. 17A through 17I illustrate tomographic imaging lung functioning visualization output forms that provide a spatial representation of a thorax to facilitate visualization of lung functioning within specific localized regions of a patient thorax. Tomographic lung function visualization output forms depict spatial information of a thorax. Under the control of signal processing circuit 200 current-voltage sensor array 100 in one embodiment is activated to inject current through a specific electrode of current-voltage sensor array 100, and voltages can be measured at each electrode of array 100. A location of current injection can be changed and the voltages measured again. The process can be continued until current has been injected from a plurality of different locations. Signal processing circuit 200 can process an obtained signal from current-voltage sensor array 100 to output a spatial representation of a thorax by output of a tomographic lung function visualization output form.

In FIG. 17J there is shown an impedance change lung function visualization output form having multiple plots 1702, 1704, 1706 corresponding to different spatial regions of a thorax. By depicting impedance changes at different spatial regions of a thorax the lung function visualization output form of FIG. 17J can facilitate visualization of lung functioning within specific localized regions of a patient thorax. Plot 1702 illustrates a lung function visualization output form for a heart region of a thorax. Plot 1704 illustrates a lung function visualization output form for a left region of a thorax. Plot 1706 illustrates a lung function visualization output form for a right lung region of a thorax. Under the control of signal processing circuit 200, the output form of FIG. 17J in one embodiment is provided by processing an obtained signal attributable to current injected or voltage applied through a select localized region of a thorax.

It will be understood that remaining output forms set forth herein provided by processing including processing of an output signal of a current-voltage sensor array 100, namely the output forms as set forth in FIGS. 3-6, 9, 11-13 can be adapted to output information of a localized region of a thorax, e.g., by appropriate control of an excitation signal and/or appropriate filtering of an obtained output signal of a current-voltage sensor array 100. One or more of the output forms of FIGS. 3-6, 9, 11-13 can facilitate visualization of lung functioning within specific localized regions of a patient thorax.

In a method for providing care to a patient, a patient in one embodiment is intubated and placed on a ventilator machine. During the time that a patient is intubated and placed on a ventilator machine, a spirometer derived output such as the flow volume change waveform lung function visualization output form of FIG. 7, the flow volume change rate waveform lung function visualization output form of FIG. 8, the spirometer signal derived flow volume loop as shown in FIG. 10 and/or the respiratory index lung function visualization output forms of FIG. 15 or FIG. 16 can be provided. However, once the patient is extubated, the spirometer signal is no longer active, and hence spirometer signal derived lung function visualization output forms are no longer provided. With features set forth herein, one or more lung function visualization output form can be provided without a spirometer signal being available.

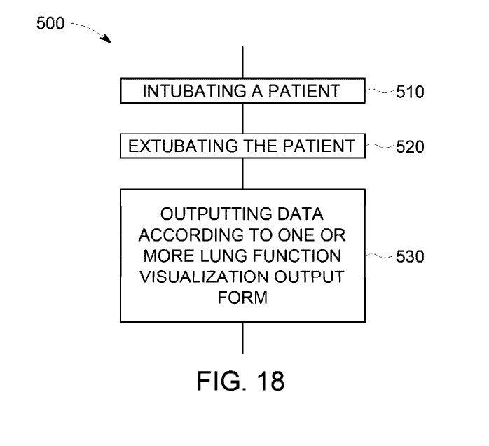

Method 500 is illustrated with reference to the flow diagram of FIG. 18. At block 510 a patient can be intubated. In one embodiment, the intubating of a patient at block 510 can be accompanied by applying a current-voltage sensor array 100 to the patient. In one embodiment, with the patient intubated, a method can include outputting to an output device 220 viewable by caregiver one or more of a spirometer signal derived lung function visualization output form or a current-voltage sensor array signal derived lung function visualization output form. At block 520 a patient can be extubated. Performance of block 520 can include the patient remaining equipped with a current-voltage sensor array 100. By applying a current-voltage sensor array 100 at block 520 and by providing current-voltage sensor array 100 so that a patent remains equipped with current-voltage sensor array 100 after block 520 a signal remains available for providing a lung function visualization output form as set forth herein after performance of block 520.

Referring further to the method as shown in FIG. 18, a method can include at block 530, subsequent to the extubation, outputting current-voltage sensor array signal derived data, which data can be derived by signal processing circuit 200 using a signal obtained from current-voltage sensor array 100. Signal processing circuit 200 can output data to an output device 220. Signal processing circuit 200 can output a lung function visualization output form as set forth herein. For example the output data in one embodiment is in one or more of a lung function visualization output form as set forth herein, e.g., the impedance change waveform lung function visualization output form of FIG. 3, the impedance change rate waveform lung function visualization output form of FIG. 4, the power change waveform lung function visualization output form as set forth in FIG. 5, the power change rate waveform lung function visualization output form of FIG. 6, the flow volume loop lung function visualization output form as shown in FIG. 9, and/or the respiratory index lung function visualization output form as shown in FIG. 12, the lung function visualization output form as shown in FIG. 13, the tomographic spatial imaging lung function visualization output form as shown in FIG. 17A-17I, and or the region specific waveform lung function visualization output forms as shown in FIG. 17J. Using a spatial imaging and/or a region specific visualization output form as shown in FIGS. 17A-17J or FIGS. 3-6, 9, 11-13 (adapted to provide information of a localized region) can be particularly helpful. For example an output form providing overall lung function information may indicate that patient lungs are functioning normally while in reality there is a deficiency in functioning in one specific area. Providing an output form as shown by the exemplary forms of the FIGS. 17A-17J can identify lung function deficiencies in a localized region of a thorax and accordingly can assist identification of scenarios in which re-intubation is appropriate.

By viewing data which can be output by signal processing circuit 200 based on a processing of a signal of a current-voltage sensor array 100, a caregiver can monitor a lung functioning of a patient after a time that the patient has been extubated. The patient can be in state without a breathing tube and can have no capacity to activate a spirometer. The lung function visualization output forms as set forth herein can provide detailed information regarding a patient's lung functioning that has not been possible via alternative non-invasive technologies.

Apparatus 10 in one embodiment is configured so that an output of apparatus 10 is based on a processing of a signal of current-voltage sensor array 100, e.g., based on a processing of a signal of current-voltage sensor array 100 without being based on a signal of a spirometer or alternatively based on a process of a signal of a current-voltage sensor array 100 and based on a processing of a signal of a spirometer. Apparatus 10 in one embodiment is configured so that an output of apparatus 10 is based on a present signal of current-voltage sensor array 100 and based on a present signal of a spirometer. Apparatus 10 in one embodiment is configured so that an output form of apparatus 10 is based on a present signal of current-voltage sensor array 100 and based on a prior signal of a spirometer. The prior signal can be signal of a spirometer during a prior stage of a method, e.g., a method as set forth in connection with FIG. 18. Referring to block 510 in one embodiment, a signal of a spirometer with a patient intubated can be used for calibration purposes to set baseline measurements for a patient for purposes of providing a patient-specific calibration for an output waveform, so that an amplitude of a waveform providing a lung function visualization output form corresponds to a volume and/or flow amplitude. After extubation, at block 530, a signal of a current-voltage sensor array 100 can be continued to be calibrated according to the amplitude established at block 510 (with a patient intubated and spirometer signal active).

Setting baseline measurements to provide a patient-specific calibration may be helpful in order for health care providers to properly read and interpret a current-voltage sensor signal based lung function visualization output form, as illustrated by FIGS. 3-6, 9, 11-13, 17A-17J. A patient's baseline or baseline measurements may be expressed in any set of units, depending on the specific lung function visualization output form being used. Regardless of the specific units used for any lung function visualization form, however, a baseline for a patient may be set while the patient is intubated. Changes from the baseline measurements following extubation may be observed by obtaining data via current-voltage sensor array 100, as described herein, and comparing the derived post-extubation lung function visualization output forms with the patient's baseline measurements.

Providing patient-specific calibration in one embodiment is performed on an ongoing and continuously updated basis while a patient is intubated, as a spirometer is available for measurement while the patient is intubated, and while a current-voltage sensor array 100 is applied to the patient. Accordingly, an output lung function visualization output form in one embodiment is based on a present signal of a current-voltage sensor array and a present signal of a spirometer. While the spirometer is actively measuring volume and flow for each time period, the current-voltage sensor array 100 may be used to determine the impedance and/or power, corresponding to each time period. This correspondence allows for calibration of volume and flow values to impedance or power, as continuously updated values for the specific patient. Amplitude of an output form in one embodiment is adjusted based on the determined calibration values and the calibration values can be continued to be used to adjust amplitude of an output form after a patient is extubated. In a current-voltage sensor array signal based lung function visualization output form, as illustrated by FIGS. 3-6, 9, 11-13, 17A-17J, the impedance, impedance change, power, power change units of the Y axis can be replaced for further labeled with volume or flow units for emphasis that impedance or power related parameters are being used to represent volume or flow information.

After the patient is extubated, the derived calibrations derived during the time that a patient was intubated (based on a prior signal of a spirometer) may be used, e.g., to convert impedance or power values to volume values (for a power change waveform or impedance over time data form), to convert the impedance derivative or power derivative values into flow (for a flow volume loop), and/or to convert the respiratory index breaths/ohms/minute into breaths/volume/minute. Patient calibration may be carried out at the “global” level (i.e., for all electrode apparatus 110 of the current-voltage sensor array 100), and such “global” calibration may then be used following extubation to obtain values for specific regions.

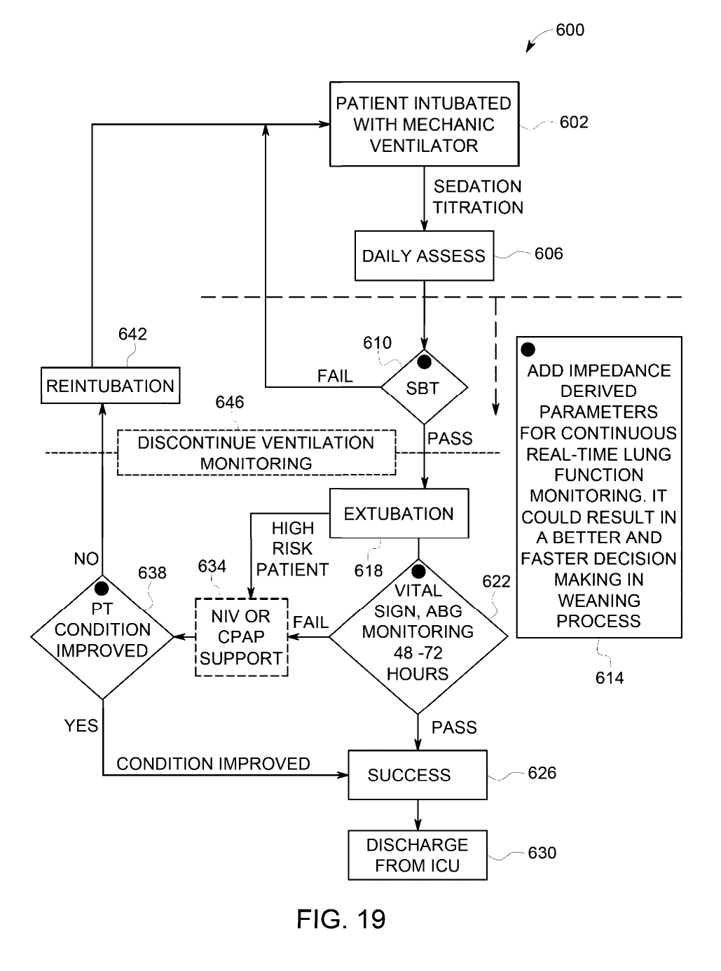

FIG. 19 illustrates one embodiment of a further application of a current-voltage sensor array 100, in which a patient may be assessed for weaning from mechanical ventilation and subsequent continued monitoring of lung function for either eventual patient discharge or, if needed, re-intubation of the patient. In a method 600 for providing care to a patient can include assessing whether a patient is ready to be weaned from mechanical ventilation, a spontaneous breathing trial (SBT) at block 610 can be performed. An SBT may involve, for example, temporarily switching the patient's tubing supply from the ventilator to a continuous oxygen supply, or may include reducing pressure of the air supply below a level at which the patient should be able to breathe independently if ready for extubation. Prior to performance at block 610 a patient can be intubated (block 602) and a daily assessment can be performed (block 606). Although a failed SBT may be injurious to a patient who is not ready to be extubated, delaying an SBT and delaying the eventual extubation of a patient may be even more injurious to the patient. It may thus be desirable to not only perform an SBT and extubate a patient as soon as feasible, it may also be desirable to properly assess the likelihood that the SBT will succeed for a particular patient to lead to extubation. As the output forms of FIGS. 3-6, 9, 11-13, 17A-17J illustrate, the impedance-derived (or power-derived or otherwise current-voltage sensor array derived) parameters that may be obtained from current-voltage sensor array 100 may be used at block 614 as part of the assessment of a patient's likelihood for passing an SBT. As explained previously the output forms of FIGS. 3-6, 9, 11-13, 17A-17J can be based on a signal of current-voltage sensor array 100 and based on a signal of a spirometer as a present signal or a past signal.

As indicated by block 646 a patient can be disconnected from a ventilator machine. When a patient is intubated a spirometer signal can be active and a patient can be monitored using a spirometer signal derived output form, e.g., as shown in FIG. 7-8, FIG. 10 and/or FIG. 14-16. In one example, as discussed above, current-voltage sensor array 100 can be used to produce an respiratory index (e.g., RSBI) lung function visualization output form for a patient, as illustrated in FIGS. 12 and 13, while the patient is intubated and being assessed for weaning from mechanical ventilation and/or after a patient is extubated. Generally several factors can be assessed to determine a patient's readiness for extubation, and in one particular embodiment the patient should have a RSBI value of less than 105 breaths/liter/min. Current-voltage sensor array 100 may thus be used to produce an RSBI lung function visualization output form (FIG. 13) for a patient to determine the patient's likelihood of passing an SBT, and a health-care provider may more quickly assess when a patient is likely to be ready for weaning from mechanical ventilation. Other visualization output forms based on a processing of a signal or current-voltage sensor array 100 can also or alternatively be produced, e.g., as shown in FIGS. 3-6, 9, 11-13, 17A-17J. when a patient is disconnected from a ventilator machine.

Using a spatial imaging and/or a region specific visualization output form as shown in FIGS. 17A-17J or FIGS. 3-6, 9, 11-13 (adapted to provide information of a localized region) can be particularly helpful. For example an output form providing overall lung function information may indicate that patient lungs are functioning normally while in reality there is a deficiency in functioning in one specific area. Providing an output form as shown by the exemplary forms of the FIGS. 17A-17J can identify deficiencies in a functioning of a localized region of a thorax and accordingly can assist identification of scenarios in which re-intubation is appropriate.

Continuing the process flow illustrated in (block 618) FIG. 19, once a successful SBT has been performed, the patient can be extubated and may continue to be monitored for improvement in lung functioning. As further illustrated in FIG. 19, current-voltage sensor array 100 may continue to be used to monitor the patient's lung functioning over a time period, e.g., a 48 to 72 hour period at block 622 and to determine if the patient may require further non-intubation interventions to improve lung-functioning, such as by application of continuous positive airway pressure (CPAP) (block 634) or other methods. A patient may be re-intubated (block 642) if monitoring of generated lung function visualization output forms, as described above, e.g., FIGS. 3-6, 9, 11-13, 17A-17J based on an obtained signal of a current-voltage sensor array 100 indicates that the patient's lung-functioning is deteriorating (block 638). Or, if the patient's functioning improves (block 626), the patient may be discharged (block 630) once lung-functioning, as derived from measures by current-voltage sensor array 100, is deemed adequate for discharge from care.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Forms of the term “based on” herein encompass relationships where an element is partially based on as well as relationships where an element is entirely based on. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure. It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the disclosure may include only some of the described embodiments. Accordingly, the invention is not to be seen as limited by the foregoing description, but is only limited by the scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An apparatus comprising:
circuitry operative to obtain a signal of a current-voltage sensor array, the current-voltage sensor array adapted to surround a thorax of a patient;
wherein the apparatus is operative to output data in one or more lung function visualization output form based on the signal of a current-voltage sensor array, the one or more lung function visualization output form selected from the group consisting of a flow volume loop lung function visualization form and a respiratory index lung function visualization output form.

2. The apparatus of claim 1, wherein the apparatus is operative to output data in one or more lung function visualization output form based on the signal of a current-voltage sensor array and based on a processing of a signal of spirometer.

3. The apparatus of claim 1, wherein the apparatus is operative to output data in one or more lung function visualization output form that outputs information of a localized region of a thorax.

4. The apparatus of claim 1, wherein current-voltage sensor array is a multiple source current-voltage sensor array.

5. The apparatus of claim 1, wherein the one or more lung function visualization output form includes a flow volume loop lung function visualization output form.

6. The apparatus of claim 1, wherein the one or more lung function visualization output form includes a respiratory index lung function visualization output form.

7. The apparatus of claim 6, wherein the respiratory index lung function visualization output form is an RSBI lung function visualization output form.

8. The apparatus of claim 1, wherein the lung function visualization output form is output to a display device.

9. The apparatus of claim 1, wherein the apparatus is operative to output data in one or more lung function visualization output form based on the signal of a current-voltage sensor array and based on a present signal of spirometer.

10. The apparatus of claim 1, wherein the apparatus is operative to output data in one or more lung function visualization output form based on the signal of a current-voltage sensor array and based on a prior signal of a spirometer, wherein the one or more lung function visualization output form is calibrated based on the past signal of a spirometer.

11. A method comprising;
intubating a patient;
extubating the patient;
wherein the method includes, subsequent to the extubating, outputting data according to one or more lung function visualization output form, the one or more lung function visualization output form based on a processing of a signal obtained from a current-voltage sensor array applied to the patient.

12. The method of claim 11, wherein the one or more lung function visualization output form outputs information of a localized region of a thorax.

13. The method of claim 11, wherein the current-voltage sensor array is a multiple source current-voltage sensor array.

14. The method of claim 11, wherein the data according to one or more lung function visualization output form is calibrated using a signal of a spirometer activated by the patient.

15. The method of claim 11, wherein the data according to one or more lung function visualization output form is calibrated using a signal of a spirometer obtained during a time when the patient is intubated.

16. The method of claim 11, wherein the one or more lung function visualization output form is selected from the group consisting of an impedance change waveform lung function visualization output form, an impedance change rate lung function visualization output form, a power change waveform lung function visualization output form, a power change rate waveform lung function visualization output form, a flow volume loop lung function visualization output form, and a respiratory index lung function visualization output form.

17. The method of claim 16, wherein the one or more lung function visualization output form includes a flow volume loop lung function visualization output form.

18. The method of claim 16, wherein the one or more lung function visualization output form includes a respiratory index lung function visualization output form.

19. The method of claim 18, wherein the respiratory index lung function visualization output form is an RSBI lung function visualization output form.

20. The method of claim 11, wherein the method includes outputting the lung function visualization output form to a display device.

21. An apparatus comprising:
circuitry operative to obtain a signal of a current-voltage sensor array, the current-voltage sensor array adapted to surround a thorax of a patient;
wherein the apparatus is operative to output data in one or more lung function visualization output form based on the signal of a current-voltage sensor array and based on a processing of a signal of a spirometer.

22. The apparatus of claim 21, wherein the signal of a current-voltage sensor array and the signal of a spirometer are present signals.

23. The apparatus of claim 21, wherein the signal of a spirometer is a past signal and wherein the apparatus uses the signal of a spirometer for calibration of the one or more lung function visualization output form.

24. The apparatus of claim 21, wherein the one or more lung function visualization output form outputs information of a localized region of a thorax.

25. The apparatus of claim 21, wherein the current-voltage sensor array is a multiple source current-voltage sensor array.

26. The apparatus of claim 21, wherein the signal of a spirometer is a past signal and wherein the apparatus uses the signal of a spirometer for calibration of the one or more lung function visualization output form, wherein the one or more lung function visualization output form outputs information of a localized region of a thorax, and wherein the current-voltage sensor array is a multiple source current-voltage sensor array.
